(19) United States
(12) Patent Application Publication
(10) Pub. No.: US 2026/0340078 A1
(43) Pub. Date: Oct. 29, 2026

(54) INFORMATION PROCESSING DEVICE, CONTROL METHOD FOR INFORMATION PROCESSING DEVICE, AND INFORMATION PROCESSING SYSTEM

(71) Applicant: ASICS CORPORATION, Kobe-shi (JP)

(72) Inventors: Yosuke MIYAZAKI, Kobe-shi (JP); Ryuichiro INABA, Kobe-shi (JP)

(73) Assignee: ASICS CORPORATION, Kobe-shi (JP)

(21) Appl. No.: 19/702,435

(22) Filed: Jun. 9, 2026

Related U.S. Application Data
(63) Continuation of application No. PCT/JP2024/046022, filed on Dec. 25, 2024.

(30) Foreign Application Priority Data
Dec. 27, 2023 (JP) 2023-221373

(51) Int. Cl. G06F 17/18

(52) U.S. Cl. CPC G06F 17/18

(57) ABSTRACT

An information processing device includes: a first storage configured to store correspondence between running time and running ability in accordance with a distance traveled; a second storage configured to store lap time of running for each running activity for a plurality of running activities performed by a user to be predicted; a memory storing instructions; and a processor configured to execute the instructions to: compute a plurality of running abilities for a predetermined distance traveled for each running activity on the basis of the lap time and the correspondence; determine a representative value of running ability for each running activity on the basis of the running abilities; generate a regression model with the representative value as an objective variable and a distance traveled in a running activity at a time when the representative value has been determined as an explanatory variable; and predict a running ability of the user to be predicted to travel a predetermined distance on the basis of the regression model.

### INCORPORATION BY REFERENCE

This application is a continuation application of International Application No. PCT/JP2024/046022, filed on Dec. 25, 2024, which claims priority of Japanese (JP) Patent Application No. 2023-221373, filed on Dec. 27, 2023, the contents of which are hereby incorporated by reference in its entirety.

### BACKGROUND

### Technical Field

This disclosure relates to an information processing device, a control method for an information processing device, and an information processing system.

### Background Information

The anaerobic threshold (AT) value has been used as an indicator when a training menu for a user that performs running is created conventionally. The AT value refers to the exercise load, which is the transition point from aerobic to anaerobic exercise when the intensity of exercise is increased. Knowing the AT value of an individual enables the prediction of race records and the setting of running pace in accordance with the objectives of the training menu. For example, Japanese Unexamined Patent Application Publication No. 2016-195661 discloses computing a heart rate during exercise on the basis of a pulse wave signal and a body motion signal of a user measured at a time when the user is performing predetermined exercise; and determining, on the basis of the heart rate during exercise and lactate level information including an anaerobic threshold (AT value) and representing a relation between a heart rate and a blood lactate level of the user acquired in advance, a degree of effect of the predetermined exercise contributing to physical strength of the user.

### SUMMARY

The AT value is estimated on the basis of the relationship between the running speed and blood lactate concentration of the user, maximum oxygen uptake (VO2max), race records under maximum effort, or the correspondence between heart rate and running speed, for example. Here, values such as blood lactate concentration and VO2max need to be measured at a specialized facility, and if measurements are not performed under maximum effort, the accuracy of the measurements may be compromised.

Therefore, a simpler method to accurately predict an athletic performance of a user without needing the efforts of measurements at specialized facilities, such as AT values, has been needed.

An information processing device according to an aspect of the present disclosure includes: a first storage configured to store correspondence between running time and running ability in accordance with a distance traveled; a second storage configured to store lap time of running for each running activity for a plurality of running activities performed by a user to be predicted; a running ability computation unit configured to compute a plurality of running abilities for a predetermined distance traveled for each running activity on the basis of the lap time and the correspondence; a determination unit configured to determine a representative value of running ability for each running activity on the basis of the running abilities; a generator configured to generate a regression model with the representative value as an objective variable and a distance traveled in a running activity at a time when the representative value has been determined as an explanatory variable; and a prediction unit configured to predict a running ability of the user to be predicted to travel a predetermined distance on the basis of the regression model.

A control method of controlling an information processing device according to an aspect of the present disclosure causes an information processing device to perform the steps of: storing, in a first storage, correspondence between running time and running ability in accordance with a distance traveled; storing, in a second storage, lap time of running for each running activity for a plurality of running activities performed by a user to be predicted; computing a plurality of running abilities for a predetermined distance traveled for each running activity on the basis of the lap time and the correspondence; determining a representative value of running ability for each running activity on the basis of the running abilities; generating a regression model with the representative value as an objective variable and a distance traveled in a running activity at a time when the representative value has been determined as an explanatory variable; and predicting a running ability of the user to be predicted to travel a predetermined distance on the basis of the regression model.

An information processing system including an information processing device and a terminal of a user to be predicted according to an aspect of the present disclosure includes: a first storage configured to store correspondence between running time and running ability in accordance with a distance traveled; a second storage configured to store lap time of running for each running activity for a plurality of running activities performed by the user to be predicted, wherein the lap time is acquired from the terminal of the user to be predicted; a running ability computation unit configured to compute a plurality of running abilities for a predetermined distance traveled for each running activity on the basis of the lap time and the correspondence; a determination unit configured to determine a representative value of running ability for each running activity on the basis of the running abilities; a generator configured to generate a regression model with the representative value as an objective variable and a distance traveled in a running activity at a time when the representative value has been determined as an explanatory variable; and a prediction unit configured to predict a running ability of the user to be predicted to travel a predetermined distance on the basis of the regression model.

An aspect of the present disclosure provides an information processing device, for example, that accurately predicts an athletic performance of a user by a simpler method without needing the efforts of measurements at specialized facilities, such as AT value measurement.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 shows a schematic diagram illustrating a configuration of a training support system according to an aspect of the present disclosure,

FIG. 2 shows an example of a functional block diagram illustrating a server (information processing device), and a user terminal (communication terminal) according to an aspect of the present disclosure,

FIG. 3 shows an example of a lap time table according to an aspect of the present disclosure,

FIGS. 4A to 4C each show a schematic diagram illustrating a process of determining representative values of running ability according to an aspect of the present disclosure,

FIG. 5 shows an example of a plot graph of a data set according to an aspect of the present disclosure,

FIG. 6 shows a flowchart illustrating an example of an operation of the server according to an aspect of the present disclosure, and

FIGS. 7A and 7B each show a diagram illustrating an example of a display screen of a user terminal according to an aspect of the present disclosure.

### DETAILED DESCRIPTION

With reference to the drawings, an aspect according to the present disclosure will be described. The drawings each are an example, and the present disclosure is not limited to what is illustrated in the drawings. For example, the number of servers (information processing devices), user terminals (communication devices), database servers, and sensor devices and their size ratios, data sets (tables), display screens, and flowcharts shown in the drawings are examples, and the present disclosure is not limited to these.

### System Configuration

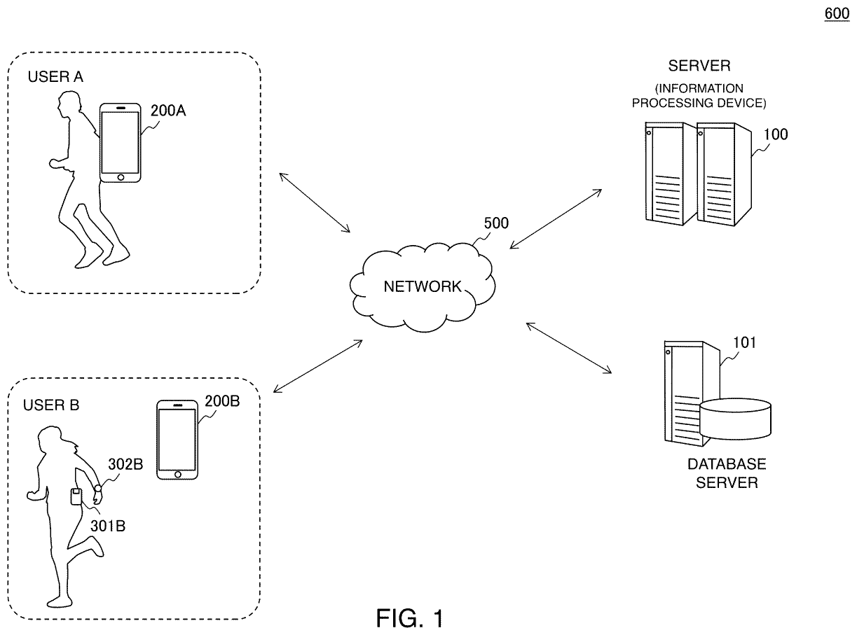

FIG. 1 shows a diagram illustrating an example of a configuration of a training support system according to an aspect of the present disclosure. A training support system 600 may be directed to an information processing system that supports training of a user (runner). According to an aspect of the present disclosure, the running ability of the user for a predetermined distance is predicted on the basis of data (information) obtained from the daily training of the user. The “running ability” corresponds to an index related to the performance and record of the user when the user runs. For example, the running ability may correspond to VDOT (pseudo VO2max), race completion time, and AT pace (running pace at an exercise load around the AT value), which will be described below, and these are correspondingly related to each other, for example, the VDOT can be converted to the race completion time or AT pace.

The term “training” as used herein may include “practice,” “discipline,” and similar activities, and may include activities voluntarily performed by an individual as well as activities performed under guidance from a specialist or a trainer.

The training support system 600 may include a server 100, a database server 101, user communication terminals (user terminals) 200 (200A, 200B), and sensor devices 301 (301B), 302 (302B). In FIG. 1, two users A and B are shown, and the respective user terminals and sensor devices are marked with the alphabetic letters “A” and “B.” However, the number of users may be greater than or equal to or less than or equal to this number, and user terminals and sensor devices may be provided in numbers corresponding to the users. Hereinafter, unless otherwise distinguished, user terminals and sensor devices will be referred to simply as user terminal 200 and sensor devices 301, 302, respectively. As will be described in detail below, the user terminal 200 and sensor devices 301, 302 may correspond to communication devices with location information acquisition functions such as global positioning system (GPS).

The server 100 is capable of performing various processes related to the training support services embodied by the training support system 600. Further, the server 100 is connected to the user terminals 200 via the network 500. The network 500 may include a wireless network and/or a wired network such as, for example, a wireless local area network (WLAN), a wide area network (WAN), integrated services digital networks (ISDNs), wireless LANs, code division multiple access (CDMA), long term evolution (LTE), LTE-Advanced, fourth generation (4G) mobile communication systems, fifth generation (5G) mobile communication systems, sixth generation (6G) and later mobile communication systems, or any combination thereof.

The server 100 also transmits and receives various data to and from the database server 101. The database server 101 stores various types of data needed for embodying functions of the training support system 600. In FIG. 1, the server 100 and database server 101 are shown connected via the network 500, but the server 100 and database server 101 may be connected by a dedicated internal network. Although FIG. 1 shows the server 100 and the database server 101 separately, the database server 101 may function as the storages (first storage and second storage) of the server 100, which will be described below. In FIG. 1, one server 100 and one database server 101 are shown respectively, but are not limited to this. That is, the functions described as provided by the server 100 may be embodied by multiple servers, or multiple database servers 101 may be provided. The server 100 may be, for example, a distributed server system that operates cooperatively by communicating over a network, or a cloud server. That is, the server 100 is not limited to physical servers, but may also include software virtual servers.

The user terminal 200 is directed to a communication terminal used by the user, and has applications installed to use the training support service (hereafter, also referred to as “training support application”). As will be described in detail below, the user terminal 200 may be able to transmit information acquired from itself and the sensor devices 301, 302 to the server 100 via the training support application.

Although FIG. 1 shows a smartphone as a user terminal 200, any terminal that is capable of embodying the functions described in the embodiments below may be used as a user terminal 200. For example, a user terminal 200 may be directed to a computer (e.g., tablet, desktop computer, laptop), handheld computing device (by way of example, not limitation, wearable device (such as eyeglass-type device (smart glasses), watch-type device (smart watch)), and smart speakers, for example. The sensor device 302 may also serve as the user terminal 200 by providing various functions of the user terminal 200 described below.

The users may include a user to be predicted as a user of which running ability is predicted for a predetermined distance, and a reference user that has running data from previous runs over the predetermined distance. The predetermined distance may be any distance the user wishes to complete, for example, a full marathon (42.195 km) and the half marathon (21.0975 km), for example. In one aspect of the present disclosure, the running data of the reference user is used to correct the prediction results of the predictive target user, thereby contributing to the improvement of the prediction accuracy. When no particular need exists to distinguish between the predictive target user and the reference user, the term “user” simply refers to both.

The user may run and wear at least one of the user terminal 200 and the sensor devices 301, 302, which have a location information acquisition function. The sensor device 301 may correspond to a motion sensor as a wearable device with communication functions, which is capable of acquiring information on a running form of the user and distance traveled by the user, and transmitting the acquired information to the user terminal 200. In addition to measuring the distance traveled, the sensor device 302 may include a vital sensor capable of detecting biometric information such as the body temperature, blood pressure, heart rate, and number of breaths per unit time of the user. However, in one aspect of the present disclosure, the device only needs to obtain information on the time and the distance traveled by the user, and acquisition of information on the running form and biometric information is not needed. That is, according to one aspect of the present disclosure, only the data acquired by holding the user terminal 200 while running is sufficient, and the attachment of motion or vital sensors is not needed.

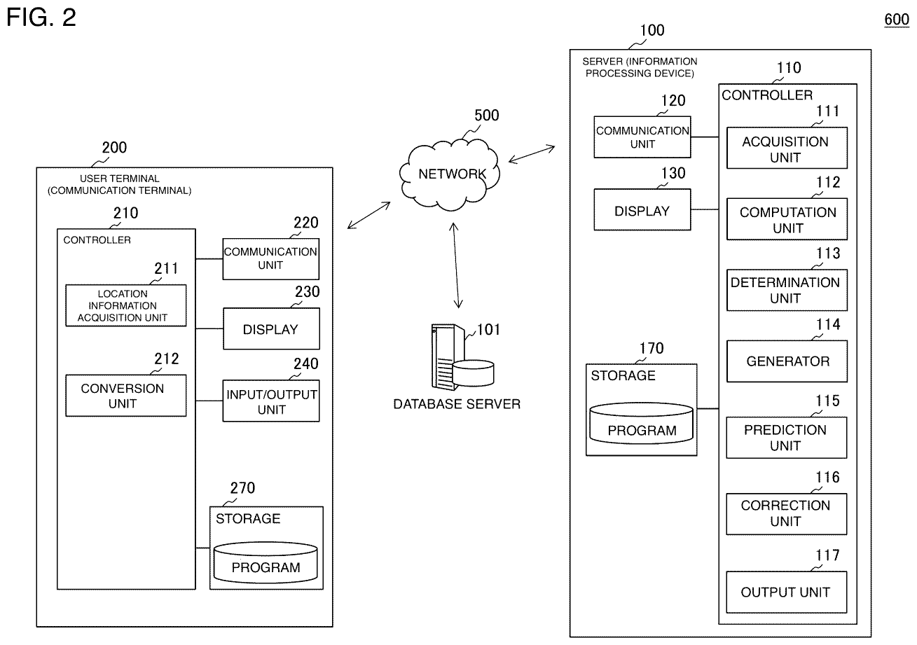

Next, with reference to FIG. 2, the hardware and functional configurations of the server 100, and user terminal 200 will be described.

### User Terminal

### (1) Hardware Configuration of User Terminal

The user terminal 200 includes a controller 210, communication unit 220, display 230, input/output unit 240, and storage 270.

The controller 210 is typically directed to a processor embodied by a central processing unit (CPU), micro processing unit (MPU), and graphics processing unit (GPU), for example. The controller 210 reads the programs stored in the storage 270 and executes the codes or instructions contained in the programs to perform the functions and methods shown in the embodiments.

The controller 210 controls the communication unit 220, display 230, and input/output unit 240. Specifically, the controller 210 controls communication between the user terminal 200, and the server 100 and sensor devices 301, 302 by the communication unit 220, and transmits and receives various data between them. For example, the controller 210 may acquire the traveled distance and traveled time as data during the running of the user as measured by the sensor devices 301, 302. The controller 210 also controls the display of data on the display 230. For example, the controller 210 may display on the display 230 the running ability, for example, predicted in running a predetermined distance. Further, the controller 210 controls the communication of various information to and from external devices via the input/output unit 240. For example, the controller 210 may transmit various types of information to the functional units in response to the input operations by the user accepted by the input device, or transmit information from the functional units to output devices not shown, such as touch panels, monitors, and speakers.

The storage 270 stores various programs and various data needed for the user terminal 200 to operate. For example, the storage 270 may store the programs for the training support application described above. The storage 270 may include, for example, flash memory, and may also include a memory (a random access memory (RAM), and read only memory (ROM), for example) that provides a work area for the controller 210. It may also temporarily store lap times, which will be described below.

The communication unit 220 is implemented as hardware such as a network adapter, communication software, and combinations thereof, and transmits and receives various data to and from the server 100, and the sensor devices 301, 302 in accordance with a predetermined protocol via the network 500.

The display 230 may correspond to a monitor that displays data in accordance with the display data written into the frame buffer, such as a touch panel or touch display, for example.

The input/output unit 240 includes input devices for various operations on the user terminal 200 and output devices for outputting the processing results processed by the user terminal 200. The input devices may include, for example, touch panels, touch displays, cameras, and microphones, and the output devices may include, for example, displays, touch panels, and speakers.

### (2) Functional Configuration of User Terminal

The user terminal 200 includes a location information acquisition unit 211 and a conversion unit 212 as functions embodied by the controller 210. The location information acquisition unit 211 acquires location information on the current location of its own terminal. The location information acquisition unit 211 acquires, for example, information on latitude and longitude of the user terminal 200 as location information of the current location of the user terminal 200 using global positioning system (GPS). The location information acquisition unit 211 may acquire location information by any method such as wireless LAN, indoor messaging system (IMES), radio frequency identifier (RFID), bluetooth low energy (BLE) (registered trademark), and geomagnetism, for example.

The conversion unit 212 converts an amount of traveling by the user per unit time acquired by the location information acquisition unit 211 into lap time for each predetermined section. The predetermined section may be, for example, but not limited to, 1,000 meters. If the user runs with the sensor devices 301, 302 worn and the sensor devices 301, 302 include a conversion function to lap time, the conversion to lap time may be performed by the sensor devices 301, 302. Alternatively, the conversion to lap time may be performed by the server 100 as will be described below.

### Server

### (1) Hardware Configuration of Server

The server 100 includes a controller 110, communication unit 120, display 130, and storage 170.

The storage 170 is typically embodied by various storage media such as a hard disc drive (HDD), solid state drive (SSD), and flash memory, and serves to store various programs and data needed for the server 100 to operate. Further, the storage 170 includes a memory (e.g., RAM, ROM) that provides a work area for the controller 110.

The controller 110 is typically a processor, embodied by a central processing unit (CPU), MPU, and/or GPU, for example. The controller 110 reads the programs stored in the storage 170 and executes the codes or instructions contained in the programs to perform the functions and methods shown in the embodiments.

The communication unit 120 is implemented by hardware such as a network adapter, communication software, and combinations of these. The communication unit 120 may transmit and receive various data to and from the user terminal 200 via the network 500 using any communication protocol.

### (2) Functional Configuration of Server

The server 100 includes an acquisition unit 111, computation unit 112, determination unit 113, generator 114, prediction unit 115, correction unit 116, and output unit 117 as functions embodied by the controller 110. In FIG. 2, the functional units that are not essential for the embodiments described hereinafter may be omitted. The function or processing of the functional units may be embodied by machine learning or AI to the extent feasible.

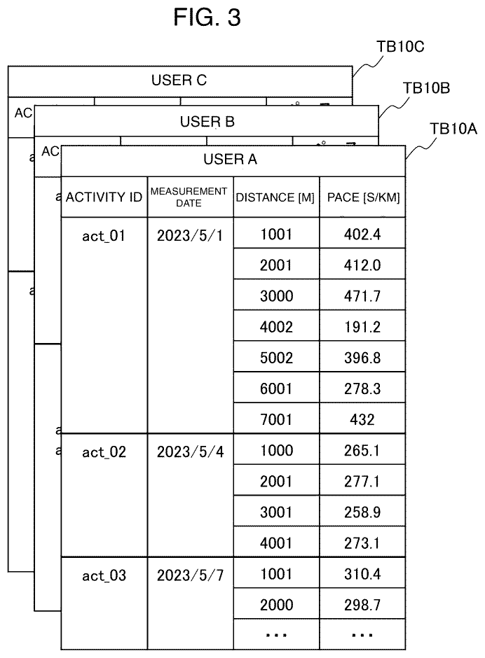

The acquisition unit 111 acquires the lap time of running for each running activity from the database server 101 for the multiple running activities performed by the user to be predicted. FIG. 3 shows an example of lap times stored in the database server 101. In FIG. 3, lap time table TB10A shows the lap time of the user to be predicted (user A), and tables TB10B and TB10C show the lap times of the reference users (user B and user C). Hereinafter, when no special distinction is needed, these lap time tables will simply be referred to as TB10. The lap time table TB10 may record lap times measured in a running activity in association with an identifier (activity ID (identifier)) that identifies the running activity. In the example of lap time table TB10, the user A has performed “7,001 m” of running activity on “2023/5/1” and the lap time for each 1,000 m is recorded. Further, the user A has performed “4,001 m” of running activity on “2023/5/4” and the lap time for each 1,000 m is recorded. These data may be computed on the basis of the data measured by the user terminal 200 and sensor devices 301, 302.

The computation unit 112 computes multiple running abilities for a predetermined distance traveled for each running activity on the basis of the lap times and the correspondence between the running time and running ability in accordance with the distance traveled. That is, the computation unit 112 functions as the running ability computation unit. Here, the “correspondence between the running time and running ability in accordance with the distance traveled” may be directed to, for example, a data set equivalent to the VDOT-based computation table proposed by Jack Daniels (hereinafter, also referred to as “Daniels' dataset”). The VDOT is a pseudo-VO2max (maximum oxygen uptake) and is an indicator of the current level of the running ability of the user.

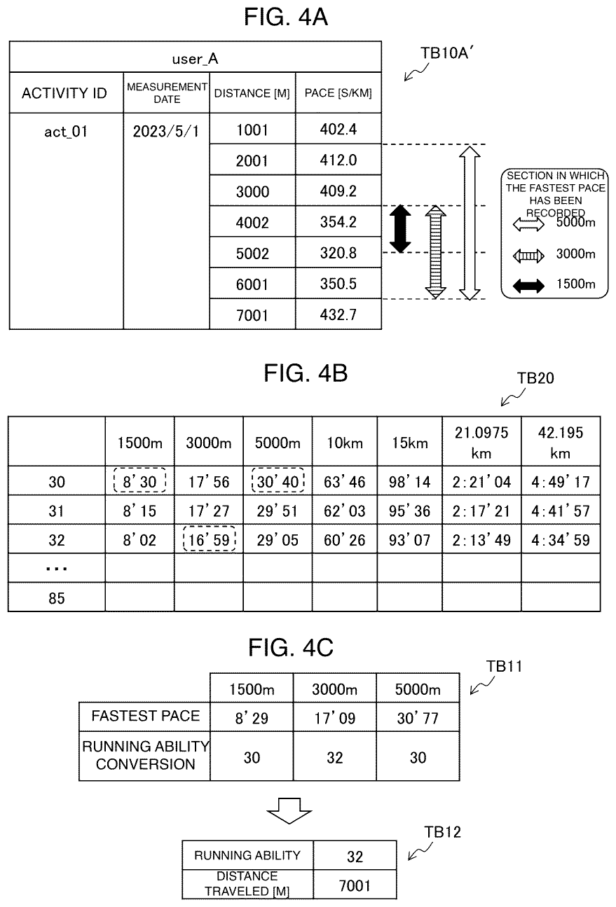

FIG. 4B shows an example of a data set indicating the correspondence between the running time and running ability in accordance with the distance traveled. According to the table TB20, theoretically, if the running ability (VDOT) is “30,” the user should be able to complete a 1,500-meter race in “8 minutes and 30 seconds” and a full marathon (42.195 km) in “4 hours, 49 minutes, and 17 seconds.” Further, if the running ability is “32,” the user should be able to complete a 3,000-meter race in “16 minutes and 59 seconds” and a full marathon in “4 hours, 34 minutes, and 59 seconds.” In one aspect of the present disclosure, the VDOT is obtained as the running ability and converted to the AT pace and completion time. The values enclosed by dashed lines in the table TB20 will be described below.

With further reference to FIGS. 4A to 4C, the processing of the computation unit 112 will be described. The table TB10A′ in FIG. 4A shows the data set of lap times for one running activity identified by the activity ID “act_01” in the lap time table TB10A for the user to be predicted (user A) shown in FIG. 3. The computation unit 112 computes the respective fastest paces for 1,500 m, 3,000 m, and 5,000 m as the predetermined distance traveled on the basis of the data in the table TB10A′. Here, 1,500 m, 3,000 m, and 5,000 m correspond to the traveled distances that have been shown to correspond to running ability in the Daniels'dataset described above. In the example in the table TB10A′, the fastest pace up to 5,000 m is computed since the total distance traveled is 7001 m. However, if the total distance traveled is greater than or equal to 10 km, the fastest pace for 10 km may be computed.

Here, the respective fastest paces for the 1,500 m, 3,000 m, and 5,000 m when the user travels have been “8 minutes 29 seconds,” “17 minutes 9 seconds,” and “30 minutes 77 seconds” as shown in the table TB11 in FIG. 4C. The computation unit 112 compares the fastest paces with the Daniels'dataset, and, for each distance traveled, sets, as a running ability of the user for the distance traveled, a running ability associated with a pace closest to the fastest pace for each distance traveled. In the examples of FIGS. 4A and 4B, a running ability “30” associated with a pace “8 minutes 30 seconds” closest to the fastest pace “8 minutes 29 seconds” for a 1,500 m distance traveled may be computed as a running ability of the user A for the 1,500 m distance traveled. In the same manner, for 3,000 m and 5,000 m, the running ability is computed as shown in the table TB11 in FIG. 4C.

The determination unit 113 determines a representative value of the running ability for each running activity on the basis of multiple running abilities. For example, the determination unit 113 may determine the maximum value of the running abilities computed by the computation unit 112 as the representative value. In the example of the table TB11 in FIG. 4C, the representative value of the running ability for the running activity identified by the activity ID “act_01” is “32.” The database server 101 associates and stores the distance traveled with the running ability for the running activity at a time when the representative value is determined. In the case of the example in FIG. 4C, the representative value “32” of the running ability for the running activity identified by the activity ID “act_01” may be associated and stored with the total distance traveled “7,001 m” as the table TB12. Since the computation unit 112 and the determination unit 113 perform the processing described above for each running activity identified by the activity ID, a data set including a representative value of the running ability and a total distance traveled is generated for each running activity.

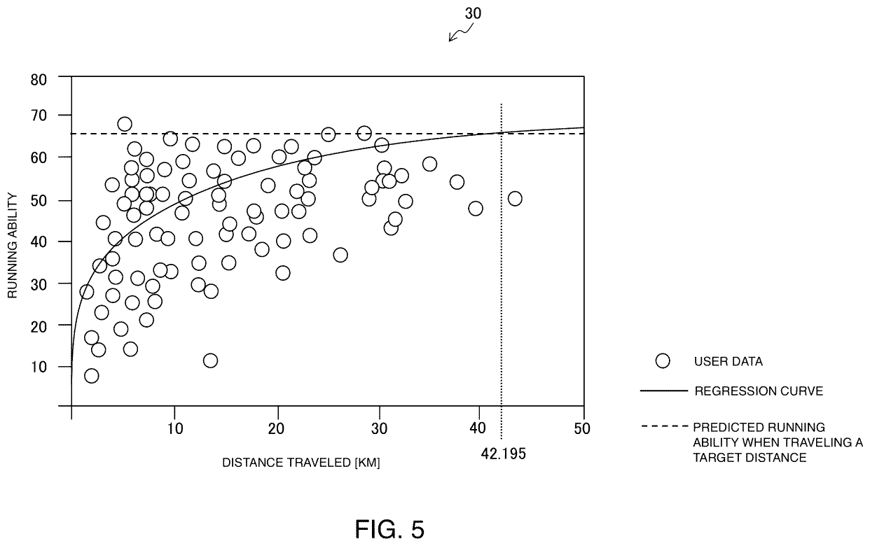

The generator 114 generates a regression model with the representative value as the objective variable and the distance traveled in the running activity at the time when the representative value has been determined as the explanatory variable. This will be illustrated with reference to FIG. 5. FIG. 5 shows an example of a graph plotting data for each running activity collected from one user, with the horizontal axis representing the distance traveled (total distance traveled) and the vertical axis representing running ability. On the basis of these data sets, the generator 114 may use existing statistical methods such as the method of least squares to generate a regression curve shown by the solid line in FIG. 5 as a regression model. For example, the generator 114 obtains the coefficients β1 and β2 when the regression curve is represented by y(x)=β1 logx+β2. The regression curve is not limited to this. The regression model may also include a regularization term (penalty term) to ensure robustness. Alternatively, the generator 114 may generate a regression model by machine learning using the data set. Multiple parameters may be used as explanatory variables, not only distance traveled.

The prediction unit 115 predicts the running ability of the user to be predicted to travel a predetermined distance on the basis of the regression model generated by the generator 114. For example, if the predetermined distance is 42.195 km, i.e., the distance of a full marathon, the prediction unit 115 may predict the running ability by substituting x=42.195 into the generated regression curve y(x).

### Control Flowchart of Server

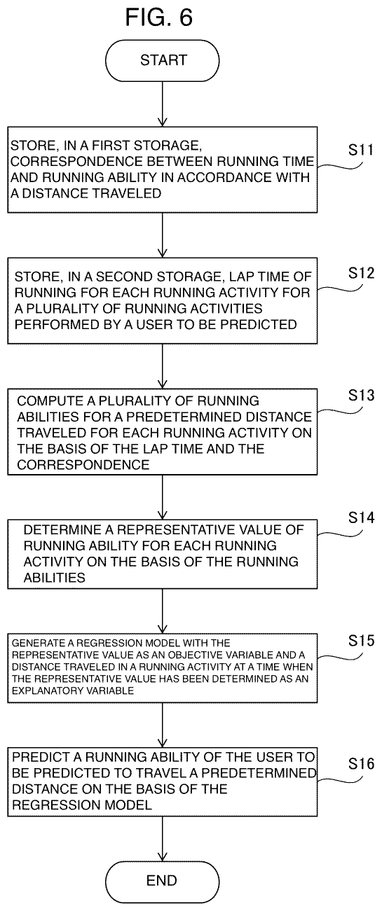

With reference to the flowchart in FIG. 6, the control method of the server 100 described above will be described. First, the first storage stores the correspondence between the running time and running ability in accordance with the distance traveled (step S11). The correspondence may be the Daniels'dataset described above shown in the table TB20 in FIG. 4B. The second storage is also caused to store the lap time of running for each running activity for the multiple running activities performed by the user to be predicted (step S12). The lap time stored in the second storage may be that stored in the lap time table TB10 in FIG. 3 described above. The first and second storages may correspond to the database server 101. The acquisition unit 111 may retrieve these data stored in the first and second storages as needed and use them in the prediction process. The computation unit 112 computes a plurality of running abilities for a predetermined distance traveled for each running activity on the basis of the lap time and the correspondence (step S13). The determination unit 113 determines a representative value of the running ability for each running activity on the basis of multiple running abilities (step S14). The generator 114 generates a regression model with the representative value as the objective variable and the distance traveled in the running activity at the time when the representative value has been determined as the explanatory variable (step S15). The prediction unit 115 predicts the running ability of the user to be predicted to travel a predetermined distance on the basis of the regression model (step S16).

As described above, according to an aspect of the present disclosure, the running ability in traveling a predetermined distance is predicted using only the lap times obtained in daily training by the user without needing measurements of biometric information such as the VO2max of the user. The lap time of the user to be predicted needs not be data measured at maximum effort, and further, it needs not be data from a long distance run such as a full marathon. This more easily predicts running ability. In addition, the prediction of running ability enables appropriate pacing during training and races, thereby improving the training efficiency.

As described above, the determination unit 113 has determined the maximum value of the running abilities computed by the computation unit 112 as the representative value. This case allows the prediction of the running ability close to the personal best of the user. However, the representative value is not limited to the maximum value, but may be the median, average, minimum, or mode, for example. If the median or mode value is used as the representative value, any bias in the distribution of user data in graph 30 may result in a prediction that is close to the actual measurement. If the minimum value is used as the representative value, the prediction may be less than the actual measurement.

Each time the user performs a run, the lap time for each run may be measured and the lap time table TB10 may be updated (accumulated). The newly accumulated lap times may then be further added to the prediction. This improves prediction accuracy.

### Displaying Prediction Results

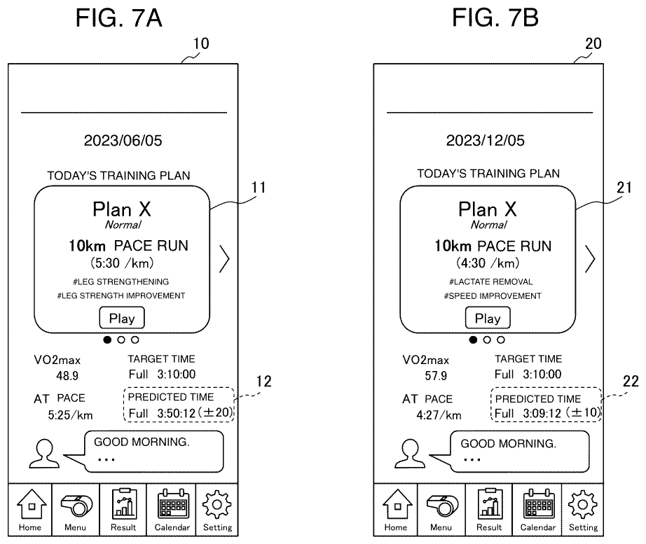

The output unit 117 may display information on the time needed to travel the predetermined distance on the user terminal 200. That is, the output unit 117 may display information on the user terminal 200 regarding the training menu recommended for the user to complete the predetermined distance, AT pace, VO2max, target time, and predicted completion time, for example, on the basis of the predicted results in running the predetermined distance. FIGS. 7A and 7B each show an example of the prediction result screen displayed on the user terminal 200. FIGS. 7A and 7B each show an example and are not limited to the manner in which it is displayed or the wording. A screen 10 in FIG. 7A shows the initial prediction result at the initial stage of the training, indicating “10 km pace run at 5:30/km” as a training menu 11. Further, the screen 10 displays, as the information on the running ability predicted at the current time, for example, a predicted time 12 needed to complete a target race. Here, the screen 10 may indicate that the predicted time 12 includes an error of plus or minus 20 minutes due to low prediction accuracy resulting from a lack of accumulated running data at an initial stage of training. Next, a screen 20 of FIG. 7B shows that a user has continued training for six months and has improved running ability, and displays, as a training menu 21, a higher-load “10 km pace run at 4:30/km.” The predicted time 22 is predicted earlier than the predicted time 12 at the initial stage of the training, and the prediction accuracy has improved to a margin of error of plus or minus 10 minutes because of the accumulation of the running data of the user.

As described above, according to an aspect of the present disclosure, information on the training ability expected to be acquired by the user is provided to the user, which is expected to have the effect of maintaining the motivation of the user.

### Correction Process

According to an aspect of the present disclosure, the prediction results for the user to be predicted may be corrected on the basis of the data acquired from multiple running activities performed by the reference user that has running data from previous runs of a predetermined distance. As shown in FIG. 3, the database server 101 further stores lap times of running (lap time tables TB10B and TB10C) for each running activity for multiple running activities performed by the reference user that has running data from previous runs of a predetermined distance. The computation unit 112, determination unit 113, generator 114, and prediction unit 115 predict the running ability of the reference user that has traveled a predetermined distance on the basis of the lap times of the running by the reference user, using the same process as for the user to be predicted described above. The correction unit 116 corrects the prediction results of the running ability of the user to be predicted by the prediction unit 115 using the prediction results of the running ability of the reference user and the actual running ability of the reference user on the basis of the running data when the reference user has run a predetermined distance in the past.

The actual running ability may be directed to a running ability that corresponds to a completion time of the reference user for a predetermined distance, referring to the Daniels'dataset described above. The correction unit 116 corrects the prediction result of the running ability of the user to be predicted using the difference (prediction error) between the prediction result of the running ability of the reference user predicted by the prediction unit 115 and the actual running ability. The correction term that minimizes the sum of the prediction errors computed for multiple reference users is computed and applied to correct the prediction results for the user to be predicted.

As described above, according to an aspect of the present disclosure, data from a reference user that has actually traveled a predetermined distance with the known actual running ability is used to correct the prediction results for the user to be predicted. This improves prediction accuracy even for users that have not actually traveled the predetermined distance.

Although the present disclosure has been described on the basis of the drawings and embodiments, it should be noted that one skilled in the art can easily make various changes and modifications on the basis of the present disclosure. Accordingly, it should be noted that these changes and modifications are included in the scope of the present disclosure. For example, functions included in respective means, respective steps, for example, may be rearranged if no logical inconsistency arises, and a plurality of means or steps may be combined into one or divided into multiple parts. In addition, the configurations shown in the above embodiments may be appropriately combined. For example, the components described as provided by the server 100 may be distributed and embodied by multiple servers. The processes described as functions of the server 100 may also be performed by the user terminal 200 and sensor devices 301, 302. Conversely, the process assumed to be performed by the user terminal 200 may be performed by the server 100 and sensor devices 301, 302.

For example, the above discussion has described the case of predicting the running ability. However, the prediction target is not limited to this, and can be, for example, lap times, AT values, and completion times of a full marathon.

The functional units of the server 100 or the information processing device 100 may be embodied by logic circuits (hardware) or dedicated circuits formed in integrated circuits (IC) chips and large scale integration (LSI), for example, or by software using a central processing unit (CPU). Each functional unit may be embodied by one or more integrated circuits, and the functions of multiple functional units may be embodied by a single integrated circuit.

The programs of each embodiment of the present disclosure may be provided as stored in a storage medium readable by the information processing device. The storage medium is directed to a “non-transitory tangible medium” capable of storing programs. The programs may include, for example, software programs and information processor programs. When the functional units of the information processing device 100 are embodied by software, the information processing device 100 functions as the acquisition unit 111, computation unit 112, determination unit 113, generator 114, prediction unit 115, correction unit 116, and output unit 117 by causing a processor to execute a program loaded into the memory.

The storage medium may include, as appropriate, one or more semiconductor-based or other integrated circuits (ICs) (for example, a field programmable gate array (FPGA) or an application specific integrated circuit (ASIC)), a hard disk drive (HDD), a hybrid hard drive (HHD), an optical disk, an optical disk drive (ODD), a magneto-optical disk, a magneto-optical drive, a floppy (registered trademark) diskette, a floppy (registered trademark) disk drive (FDD), a magnetic tape, a solid-state drive (SSD), a RAM drive, a secure digital card or drive, any other suitable storage medium, or any suitable combination of two or more thereof. The storage medium may be volatile, nonvolatile, or a combination of volatile and nonvolatile as appropriate.

The program of this disclosure may be provided to the information processing device 100 via any transmission medium (e.g., communication network, and broadcast wave) capable of transmitting the program.

The embodiments of the present disclosure can also be embodied in the form of data signals embedded in a carrier wave, in which the program is embodied by electronic transmission. The program according to the present disclosure may be implemented using, for example, scripting languages such as JavaScript (registered trademark) and Python, or programming languages such as C, Go, Swift, Kotlin, and Java (registered trademark).

It is understood by those skilled in the art that the embodiments described above correspond to specific examples of the following aspects.

[1]

An information processing device according to the present disclosure includes: a first storage configured to store correspondence between running time and running ability in accordance with a distance traveled; a second storage configured to store lap time of running for each running activity for a plurality of running activities performed by a user to be predicted; a running ability computation unit configured to compute a plurality of running abilities for a predetermined distance traveled for each running activity on the basis of the lap time and the correspondence; a determination unit configured to determine a representative value of running ability for each running activity on the basis of the running abilities; a generator configured to generate a regression model with the representative value as an objective variable and a distance traveled in a running activity at a time when the representative value has been determined as an explanatory variable; and a prediction unit configured to predict a running ability of the user to be predicted to travel a predetermined distance on the basis of the regression model.

[2]

In the information processing device of [1] above, the second storage may further store lap times of running for each running activity for multiple running activities performed by a reference user that has running data from previous runs of the predetermined distance, the running ability computation unit may compute a plurality of running abilities for a predetermined distance traveled for each running activity of the reference user on the basis of the lap times of the reference user and the correspondence, the determination unit may determine a representative value of running ability for each running activity of the reference user on the basis of the running abilities of the reference user, the generator may generate a regression model with the representative value of the reference user as an objective variable and the distance traveled in the running activity at a time when the representative value has been determined as an explanatory variable, and the prediction unit may predict a running ability of the reference user when the reference user travels a predetermined distance on the basis of the regression model of the reference user. The information processing device may further include a correction unit configured to correct a prediction result of a running ability of the user to be predicted by the prediction unit using a prediction result of the running ability of the reference user and an actual running ability of the reference user on the basis of running data at a time when the reference user has traveled the predetermined distance in a past.

[3]

In the information processing device according to [1] above, the determination unit may determine a maximum value of the running abilities computed by the running ability computation unit as the representative value.

[4]

In the information processing device according to [1] above, the determination unit may determine a median value of the running abilities computed by the running ability computation unit as the representative value.

[5]

In the information processing device according to [1] above, the determination unit may determine an average value of the running abilities computed by the running ability computation unit as the representative value.

[6]

The information processing device according to [1] above may further include an output unit configured to display, on a terminal, information on time needed to travel the predetermined distance, which has been converted from the running ability of the user to be predicted, as corrected by the correction unit.

[7]

A control method according to the present disclosure causes an information processing device to perform the steps of: storing, in a first storage, correspondence between running time and running ability in accordance with a distance traveled; storing, in a second storage, lap time of running for each running activity for a plurality of running activities performed by a user to be predicted; computing a plurality of running abilities for a predetermined distance traveled for each running activity on the basis of the lap time and the correspondence; determining a representative value of running ability for each running activity on the basis of the running abilities; generating a regression model with the representative value as an objective variable and a distance traveled in a running activity at a time when the representative value has been determined as an explanatory variable; and predicting a running ability of the user to be predicted to travel a predetermined distance on the basis of the regression model.

[8]

An information processing system including an information processing device and a terminal of a user to be predicted according to the present disclosure includes: a first storage configured to store correspondence between running time and running ability in accordance with a distance traveled; a second storage configured to store lap time of running for each running activity for a plurality of running activities performed by the user to be predicted, wherein the lap time is acquired from the terminal of the user to be predicted; a running ability computation unit configured to compute a plurality of running abilities for a predetermined distance traveled for each running activity on the basis of the lap time and the correspondence; a determination unit configured to determine a representative value of running ability for each running activity on the basis of the running abilities; a generator configured to generate a regression model with the representative value as an objective variable and a distance traveled in a running activity at a time when the representative value has been determined as an explanatory variable; and a prediction unit configured to predict a running ability of the user to be predicted to travel a predetermined distance on the basis of the regression model.

What is claimed is:

1. An information processing device comprising:
a first storage configured to store correspondence between running time and running ability in accordance with a distance traveled;
a second storage configured to store lap time of running for each running activity for a plurality of running activities performed by a user to be predicted;
a memory storing instructions; and
a processor configured to execute the instructions to:
compute a plurality of running abilities for a predetermined distance traveled for each running activity on the basis of the lap time and the correspondence;
determine a representative value of running ability for each running activity on the basis of the running abilities;
generate a regression model with the representative value as an objective variable and a distance traveled in a running activity at a time when the representative value has been determined as an explanatory variable; and
predict a running ability of the user to be predicted to travel a predetermined distance on the basis of the regression model.

2. The information processing device according to claim 1, wherein
the second storage further stores lap times of running for each running activity for multiple running activities performed by a reference user that has running data from previous runs of the predetermined distance, and
the processor is configured to execute the instructions to:
compute a plurality of running abilities for a predetermined distance traveled for each running activity of the reference user on the basis of the lap times of the reference user and the correspondence,
determine a representative value of running ability for each running activity of the reference user on the basis of the running abilities of the reference user,
generate a regression model with the representative value of the reference user as an objective variable and the distance traveled in the running activity at a time when the representative value has been determined as an explanatory variable,
predict a running ability of the reference user when the reference user travels a predetermined distance on the basis of the regression model of the reference user, and
correct a prediction result of a running ability of the user to be predicted using a prediction result of the running ability of the reference user and an actual running ability of the reference user on the basis of running data at a time when the reference user has traveled the predetermined distance in a past.

3. The information processing device according to claim 1, wherein the processor is configured to determine a maximum value of the computed running abilities as the representative value.

4. The information processing device according to claim 1, wherein the processor is configured to determine a median value of the computed running abilities as the representative value.

5. The information processing device according to claim 1, wherein the processor is configured to determine an average value of the computed running abilities as the representative value.

6. The information processing device according to claim 2, wherein the processor is further configured to execute the instructions to display, on a terminal, information on time needed to travel the predetermined distance, which has been converted from the running ability of the user to be predicted, as corrected.

7. A control method of controlling an information processing device including a memory storing instructions, and a processor, the method causing the processor of the information processing device to execute the instructions to perform the steps of:
storing, in a first storage, correspondence between running time and running ability in accordance with a distance traveled;
storing, in a second storage, lap time of running for each running activity for a plurality of running activities performed by a user to be predicted;
computing a plurality of running abilities for a predetermined distance traveled for each running activity on the basis of the lap time and the correspondence;
determining a representative value of running ability for each running activity on the basis of the running abilities;
generating a regression model with the representative value as an objective variable and a distance traveled in a running activity at a time when the representative value has been determined as an explanatory variable; and
predicting a running ability of the user to be predicted to travel a predetermined distance on the basis of the regression model.

8. An information processing system including an information processing device and a terminal of a user to be predicted, the information processing device comprising:
a first storage configured to store correspondence between running time and running ability in accordance with a distance traveled;
a second storage configured to store lap time of running for each running activity for a plurality of running activities performed by the user to be predicted, wherein the lap time is acquired from the terminal of the user to be predicted;
a memory storing instructions; and
a processor configured to execute the instructions to:
compute a plurality of running abilities for a predetermined distance traveled for each running activity on the basis of the lap time and the correspondence;
determine a representative value of running ability for each running activity on the basis of the running abilities;
generate a regression model with the representative value as an objective variable and a distance traveled in a running activity at a time when the representative value has been determined as an explanatory variable; and
predict a running ability of the user to be predicted to travel a predetermined distance on the basis of the regression model.
